(19) United States
(12) Patent Application Publication
(10) Pub. No.: US 2013/0053707 A1
(43) Pub. Date: Feb. 28, 2013

(54) SYSTEMS AND METHODS FOR UNASSISTED BLOOD PRESSURE MEASUREMENT

(71) Applicant: Jason Lee Mirisoloff, Cato, NY (US)

(72) Inventor: Jason Lee Mirisoloff, Cato, NY (US)

(73) Assignee: Welch Allyn, Inc.

(21) Appl. No.: 13/215,610

(22) Filed: Aug. 23, 2011

(51) Int. Cl. A61B 5/022

(52) U.S. Cl. 600/499

(57) ABSTRACT

A blood pressure cuff includes a substantially cylindrical sleeve configured to accept an arm of a patient. The sleeve includes an inflatable bladder having a first end, and a second end disposed opposite the first end. The sleeve also includes an elastic panel connected intermediate the first and second ends of the bladder. The blood pressure cuff also includes a tail connected to the second end of the bladder. The tail includes a longitudinal axis disposed transverse to a central axis of the sleeve.

### FIELD OF THE INVENTION

The present disclosure relates to systems and methods for blood pressure determination and, in particular, to systems and methods for unassisted blood pressure measurement.

### BACKGROUND OF THE INVENTION

The measurement of blood pressure is a common procedure used in hospitals, clinics, and other health care facilities to assist in diagnosing illnesses and monitoring patient health. In standard non-invasive blood pressure measurement practice, a patient's arterial blood pressure is measured using an inflatable sleeve, commonly referred to as a blood pressure cuff. The cuff is typically adapted to fit around a limb of the patient, and such cuffs are usually sized to fit around the patient's upper arm, between the armpit and the elbow.

In most instances, such blood pressure cuffs are wrapped around the patient's arm by a health care professional prior to obtaining a blood pressure measurement. The configuration of known blood pressure cuffs, however, makes it difficult for a patient wishing to monitor his or her own blood pressure to wrap such cuffs around his or her own arm without assistance. In particular, most known cuffs include a substantially linear inflatable bladder, and a tail section having one or more connection devices. Prior to use, the bladder must be wrapped around the patient's upper arm. The tail must then be wrapped around the deflated bladder and secured to the outer surface of the bladder prior to inflation. After securing the cuff around the upper arm, the bladder is inflated to occlude the brachial artery, the primary blood vessel in the arm carrying blood away from the heart. Although a healthcare professional may position a standard blood pressure cuff on a patient's arm relatively easily, a patient wishing to monitor his or her own blood pressure without assistance must perform each of these steps with a single hand. Such a task may be difficult to accomplish, and this difficulty may be compounded if, for example, the patient is elderly, without his or her full range of motor skills, or otherwise weakened due to injury or illness.

The exemplary embodiments of the present disclosure are directed toward overcoming the deficiencies described above.

### SUMMARY

In an exemplary embodiment of the present disclosure, a blood pressure cuff includes a substantially cylindrical sleeve configured to accept an of a patient. The sleeve includes an inflatable bladder having a first end, and a second end disposed opposite the first end. The sleeve also includes an elastic panel connected intermediate the first and second ends of the bladder. The blood pressure cuff also includes a tail connected to the second end of the bladder. The tail includes a longitudinal axis disposed transverse to a central axis of the sleeve.

In another exemplary embodiment of the present disclosure, a blood pressure cuff includes a substantially cylindrical sleeve configured to accept an arm of a patient. The sleeve includes an inflatable bladder having a first edge, a second edge disposed opposite the first edge, and a first end disposed transverse to the first and second edges. The sleeve also includes a flexible sheet connected along a portion of the first and second edges of the bladder. The sleeve further includes an elastic panel connected to the first end of the bladder. At least a portion of the elastic panel is disposed between the sheet and the bladder.

In a further exemplary embodiment of the present disclosure, a blood pressure cuff includes a substantially cylindrical sleeve configured to accept an arm of a patient. The sleeve includes an elastic panel, and an inelastic panel connected to the elastic panel along a width of the sleeve. The blood pressure cuff further includes a flexible sheet connected to the inelastic panel, and an inflatable bladder connected to the sleeve. The bladder includes a longitudinal axis disposed transverse to a central axis of the sleeve. The blood pressure cuff also includes a tail connected to the bladder. The tail includes a longitudinal axis disposed collinear with a longitudinal axis of the bladder and transverse to a central axis of the sleeve.

In another exemplary embodiment of the present disclosure, a blood pressure cuff includes a substantially cylindrical sleeve including a cover and an inflatable bladder disposed within the cover. An exterior of the cover has an outer wall and in inner wall, where the outer wall includes a first connector and the inner wall is configured to accept an arm of a patient. The blood pressure cuff also includes a tail connected to the sleeve. The tail includes a second connector releasably attachable to the first connector.

### BRIEF DESCRIPTION OF THE DRAWINGS

FIG. 1 illustrates a blood pressure cuff according to an exemplary embodiment of the present disclosure.

FIG. 2 illustrates a blood pressure cuff according to another exemplary embodiment of the present disclosure.

FIG. 3 illustrates a blood pressure cuff according to a further exemplary embodiment of the present disclosure.

FIG. 4 is a partial cross-sectional view of the exemplary blood pressure cuff shown in FIG. 3.

FIG. 5 illustrates a blood pressure cuff according to yet another exemplary embodiment of the present disclosure.

### DETAILED DESCRIPTION

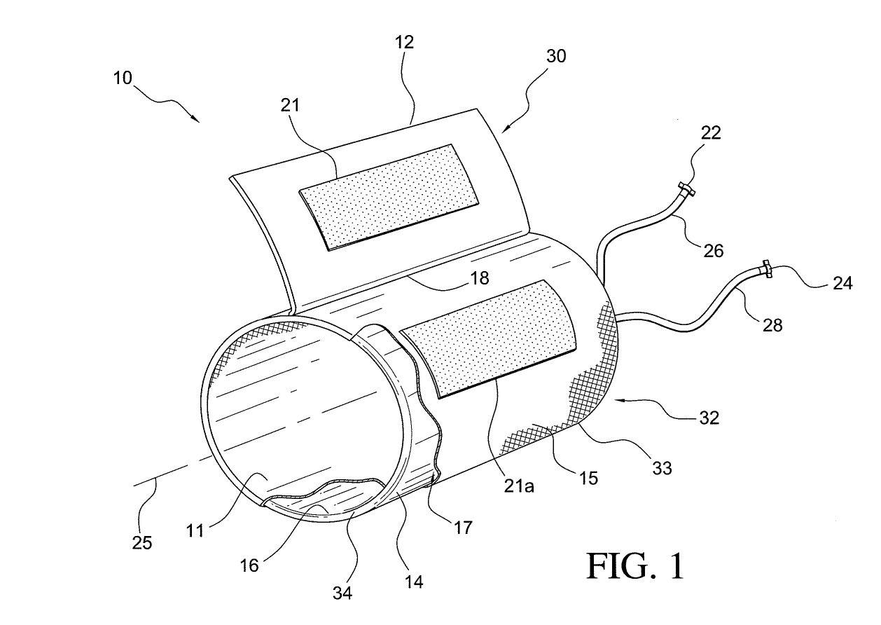

FIG. 1 illustrates an exemplary blood pressure cuff 10 of the present disclosure. The blood pressure cuff 10 includes a substantially cylindrical (i.e., substantially tube-like), flexible, and/or expandable sleeve 32, and a connection portion 30 connected to the sleeve 32. While each of the exemplary sleeves 32 of the present disclosure may be substantially cylindrical in shape, such substantially cylindrical sleeves 32 may also lay substantially flat and/or may maintain other shapes/configurations when not worn by a patient due to the flexibility, elasticity, and/or other characteristics of such sleeves 32. In the embodiment shown in FIG. 1, the sleeve 32 includes a flexible elastic cover 33 and at least one inflatable bladder 34 disposed within the cover 33.

The bladder 34 includes an outer surface 14 and an inner surface 16 disposed opposite the outer surface 14. In the exemplary embodiment illustrated in FIG. 1, the bladder 34 may be formed through a blown film extrusion process. In such a process, the bladder 34 may be formed in the shape of a substantially cylindrical tube. The tube may then be folded onto itself and sealed to form a substantially fluid-tight bladder 34.

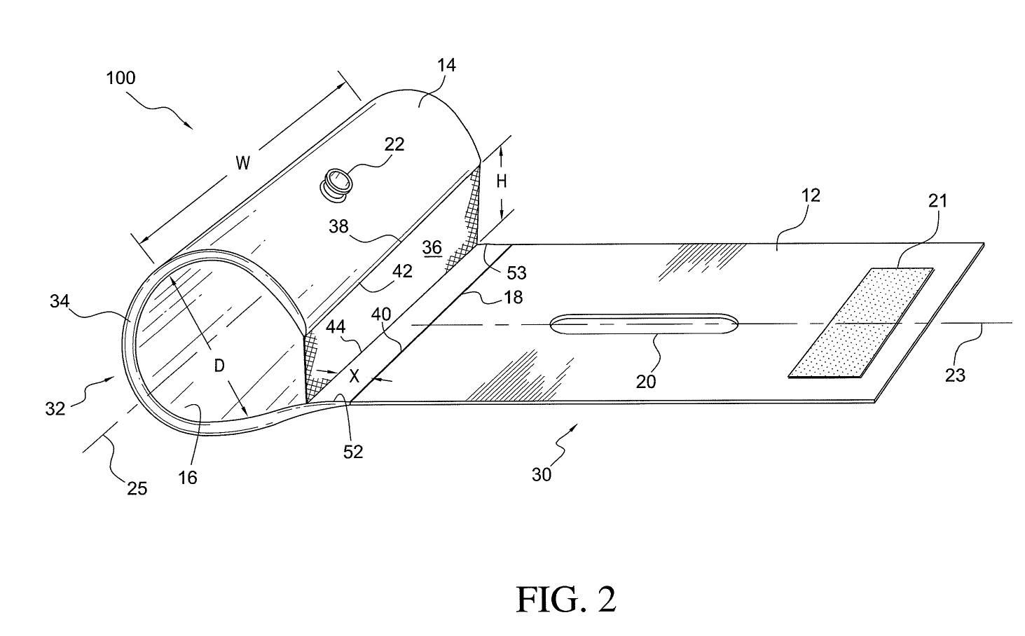

In further exemplary embodiments, the bladder 34 may be formed from a single piece of material, and the piece of material may be folded to form the outer surface 14 and the inner surface 16. In such exemplary embodiments, a remaining perimeter of the piece of material may be sealed opposite the fold through heat sealing, ultrasonic or RF welding, adhering, and/or other processes to form a substantially fluid-tight bladder 34. In additional exemplary embodiments, the bladder 34 may be formed from separate pieces of material. In such embodiments, a piece of material forming the outer surface 14 of the bladder 34 may be sealed to a separate piece of material forming the inner surface 14 about respective perimeters of the separate pieces. These separate pieces of material may be sealed through any of the processes described above. For example, the bladder 34 illustrated in FIGS. 2-5 of the present disclosure may be formed from separate pieces of material sealed along peripheral edges thereof to form a substantially fluid-tight bladder 34.

The bladder 34, cover 33, and/or other components of the blood pressure cuffs described herein may be formed from any medically approved material known in the art. Such materials may be highly flexible, durable, and suitable for contact with, for example, the skin of a patient. Such materials may also be tear-resistant, fluid-impermeable, and/or recyclable. Such materials may include, for example, paper, cloth, mesh, plastics, and/or polymers such as polypropylene or polyethylene. In still further exemplary embodiments, such materials may be coated and/or otherwise treated with one or more additives that cause the material to become biodegradable within a desired time interval (e.g., within 2 to 3 months).

For example, the cover 33 shown in FIG. 1 may be made from any of the materials discussed above, and in additional exemplary embodiments, the cover 33 may be made from one or more flexible elastic fabrics or cloth-like materials such as spandex, lycra, nylon, and the like. An exterior of such a cover 33 may include an inner wall 11 and an outer wall 15, and the walls 11, 15 of the cover 33 may form an expandable cavity 17 therebetween. In exemplary embodiments, the cavity 17 may be substantially cylindrical in shape, and may be sized to accept the inflatable bladder 34 therein. FIG. 1 illustrates an exemplary embodiment of the cover 33 with a portion removed to expose the cavity 17 and the outer and inner surfaces 14, 16 of the bladder 34. Thus, the cavity 17 and/or other components of the cover 33 may be configured to maintain the bladder 34 in a substantially cylindrical shape while the bladder 34 is disposed therein. In exemplary embodiments, one or more portions of the cover 33 may be stitched, sewn, and/or otherwise joined to form a substantially closed cavity 17 enclosing the bladder 34.

The blood pressure cuff 10 may further include a port 22 fluidly connected to the bladder 34. As shown in FIG. 1, in exemplary embodiments, the blood pressure cuff 10 may include separate first and second ports 22, 24 fluidly connected to the bladder 34. In such embodiments, the bladder 34 may include separate respective orifices (not shown) configured to accept at least a portion of tubes 26, 28 fluidly and/or mechanically connected to each respective port 22, 24. For example, at least a portion of each tube 26, 28 may be fluidly connected to a respective orifice of the bladder 34 so as to provide a fluid passage from each respective port 22, 24 into and out of the bladder 34. In the exemplary embodiment of FIG. 1, the first port 22 may be fluidly connected to the bladder 34 via the first tube 26 and the second port 24 may be fluidly connected to the bladder 34 via the second tube 28. In such an exemplary embodiment, the tubes 26, 28 may pass through the cover 33 to connect with the bladder 34.

In exemplary embodiments, each port 22, 24 may comprise an open-ended cylindrical cavity. A combination of bulbs, pumps, adapters, fittings, valves, nozzles, tubes, hoses, gages, and/or other known flow control, inflation, and/or measurement devices may be mechanically and/or fluidly connectable to each port 22, 24. For example, each port 22, 24 may facilitate connection with one or more hand operated inflation bulbs by way of a releasable fitting. A portion of each port 22, 24 may include, for example, a circumferential shelf, flange, ridge, and/or other like structure to facilitate such a connection.

In additional exemplary embodiments of the present disclosure, such as the embodiments shown in FIGS. 2-5, a blood pressure cuff may include a single port 22, and the port 22 may be sealed to the bladder 34 about a perimeter of an orifice formed in the bladder 34. In such exemplary embodiments, the port 22 may be sealed to either the outer surface 14 or the inner surface 16 of the bladder 34, and such a port 22 may be sealed to the bladder 34 by, for example, heat sealing, ultrasonic or RF welding, adhering, and/or any other process known in the art. Additional details concerning such exemplary port designs are provided in co-owned U.S. Pat. No. 6,422,086, entitled “Low Profile Pressure Measuring Device,” the entire disclosure of which is incorporated herein by reference.

The connection portion 30 may be connected to the sleeve 32 along a seam 18 through heat sealing, ultrasonic or RF welding, stitching, sewing, adhering, and/or any other like process. In additional exemplary embodiments, the connection portion 30 may be alined from the same piece and/or pieces of material used to form the cover 33. Moreover, in the exemplary embodiments of FIGS. 2-5, the connection portion 30 may be formed from the same piece of material as either the outer surface 14 or the inner surface 16 of the bladder 34. In such exemplary embodiments, the seam 18 may be omitted.

The connection portion 30 may include a tail 12 and a connector 21 disposed on the tail 12. The connector 21 may include any known adhesive, hook, loop, fastener, clip, snap, tie, and/or other releasable connection device. In further exemplary embodiments, the connector 21 may include one or more patches of Velcro® connected to the tail 12. As shown in FIG. 1, a first connector 21a may be connected to the outer wall 15 of the cover 33 and a second connector 21 may be connected to the tail 12. In such exemplary embodiments, the first and second connectors 21a, 21 may comprise mating Velcro® patches and/or other like patches of releasably attachable inelastic material. In the exemplary embodiment of FIG. 1, such connectors 21a, 21 may comprise inelastic portions of the cover 33 and/or the tail 12, respectively, and such connectors 21a, 21 may restrict local expansion of the flexible elastic cover 33 during inflation of the bladder 34. The tail 12 may have any length suitable for securing the blood pressure cuff 10 to the arm of a patient, and in exemplary embodiments, the tail 12 may have a length sufficient to overlay substantially the entire outer circumference of the blood pressure cuff 10. In additional exemplary embodiments, the length of the tail 12 may be greater than the outer circumference of the blood pressure cuff 10. In such exemplary embodiments, the connector 21a may be relocated from the position illustrated in FIG. 1 (on a first side of the seam 18) to a position on the opposite side (a second side) of the seam 18. In still further exemplary embodiments, the connector 21a may be relocated to the tail 12 to facilitate securing the blood pressure cuff 10 to the patient's arm. In such an embodiment, the connector 21a may be disposed on a first side of the tail 12 and the connector 21 may be disposed on a second side of the tail 12 opposite the first side. In further exemplary embodiments, such as the embodiments illustrated in FIGS. 2-5, the connector 21 may be disposed on an underside of the tail 12, and the connector 21 may be configured to releasably attach to the outer surface 14 of the bladder 34 and/or a topside of the tail 12. Such attachment may assist in securing the blood pressure cuff to the arm of the patient. In the embodiments shown in FIGS. 2-5, the connector 21a described above may be omitted.

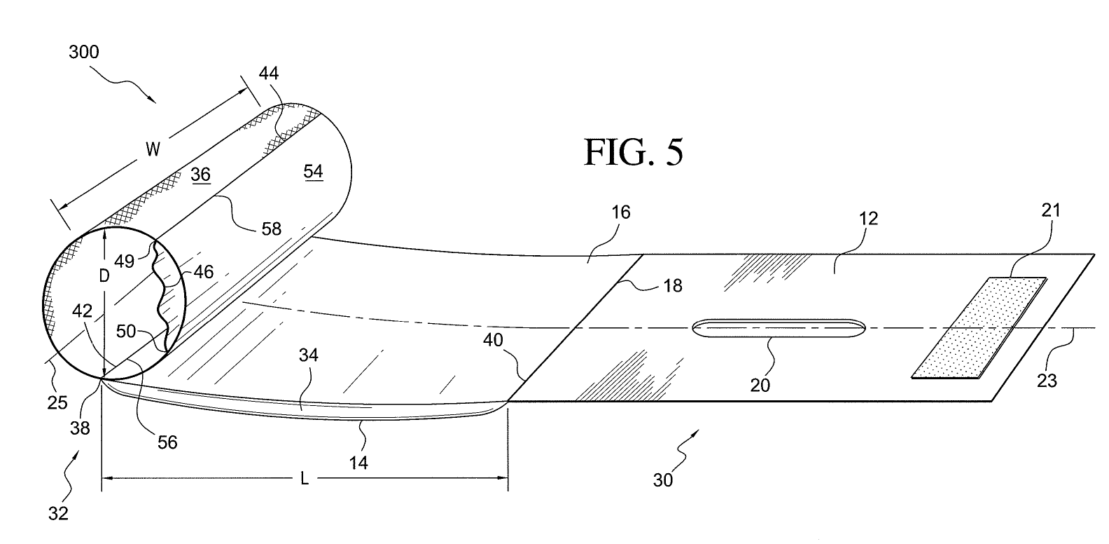

Additionally, in the embodiments of FIGS. 2-5 the connection portion 30 may include at least one slot 20. The slot 20 may extend along a longitudinal axis 23 of the tail 12, and in an exemplary embodiment, the slot 20 may be aligned with the port 22. The slot 20 may have a width transverse to the longitudinal axis 23 that is sized to accept passage of the port 22 therethrough. The slot 20 may also have a longitudinal length that limits the effective circumference of the sleeve 32. In exemplary embodiments, the slot 20 may have a length between approximately 5 inches and approximately 6 inches. Such a length may yield a maximum sleeve circumference of approximately 15 inches and a minimum sleeve circumference of approximately 9 inches. As a result, the range of arm sizes (i.e., circumferences) on which the blood pressure cuff 10 can be used may be limited by the length of the slot 20. In exemplary embodiments, the tail 12 may be configured to encircle at least 75 percent of the bladder 34 when the blood pressure cuff is wrapped around and/or otherwise secured to an arm of the patient. In further exemplary embodiments, the tail 12 may be configured to encircle greater than 100 percent of the bladder 34 when the blood pressure cuff is secured to the patient's arm, and in such exemplary embodiments, the tail 12 may have a length greater than a length L (FIG. 5) of the bladder 34. In exemplary embodiments, the length L of the bladder 34 may be between approximately 10 inches and approximately 14 inches. In further exemplary embodiments, the length L may be equal to approximately 12 inches.

While the exemplary sleeve 32 illustrated in FIG. 1 includes a cover 33 and an inflatable bladder 34 disposed within a cavity 17 of the cover 33, in additional exemplary embodiments, the inner surface 16 of the exterior of the bladder 34 may comprise an inner wall of the sleeve 32 and the outer surface 14 of the bladder 34 opposite the inner surface 16 may comprise an outer wall of the sleeve 32. In such embodiments, the cover 33 may be omitted. For example, as shown in FIGS. 2-4, a blood pressure cuff 100, 200 may include a substantially cylindrical sleeve 32 configured to accept an arm of a patient, and at least a portion of the inner and outer walls of the sleeve 32 may be formed by the respective inner and outer surfaces 16, 14 of the inflatable bladder 34. Such sleeves 32 may also include at least one elastic panel 36 connected to the bladder 34.

As shown in at least FIG. 2, the bladder 34 may include a first end 38 and a second end 40 disposed opposite the first end 38. The bladder 34 may also include a first edge 52 disposed substantially transverse to the first and second ends 38, 40. In addition, the bladder 34 may include a second edge 53 opposite the first edge 52, and the first and second edges 52, 53 may be disposed substantially parallel to the longitudinal axis 23 of the tail 12. The ends 38, 40 and edges 52, 53 of the bladder 34 may be fluidly sealed through any of the processes described above

As shown in FIGS. 1-5, the sleeves 32 of the exemplary blood pressure cuffs described herein may remain in a substantially cylindrical configuration while removed from the patient's arm. This substantially cylindrical configuration of the sleeve 32 may be maintained while the sleeve 32 encircles the patient's aim before, during, and after inflation of the bladder 34. In such exemplary embodiments, the substantially cylindrical sleeve 32 may include a central axis 25. Thus, as shown in FIG. 2, the first and second ends 38, 40 of the bladder 34 may be disposed substantially parallel to the central axis 25 of the sleeve 32, while the first and second edges 52, 53 may be disposed substantially transverse to the central axis 25. In addition, the longitudinal axis 23 of the tail 12 may be disposed substantially transverse to the central axis 25 of the sleeve 32.

The elastic panel 36 may be made from lycra, spandex, nylon, and/or any of the other materials described above with respect to the cover 33, and may be connected to the bladder 34 through stitching, sewing, heat sealing, ultrasonic or RF welding, adhering, and/or other known processes. In an exemplary embodiment, the elastic panel 36 may be a cloth and/or fabric mesh configured to add flexibility to the sleeve 32. For example, the elastic panel 36 may facilitate disposal of the sleeve 32 on and/or removal of the sleeve 32 from a patient's arm by enabling a temporary localized increase in, for example, a diameter D and/or circumference of the sleeve 32. Such an increase in the diameter D and/or circumference of the sleeve 32 may facilitate moving the sleeve 32 along the patient's arm over, for example, an elbow and/or wrist of the patient.

The elastic panel 36 may have any width and/or height useful in facilitating a temporary increase in the dimensions of the sleeve 32. For example, in the exemplary embodiment of FIG. 2, the elastic panel 36 may have a height H between approximately 1 inch and approximately 4 inches. In further exemplary embodiments, the height H may be equal to approximately 1.5 inches. Such heights H may be, for example, between approximately one eighth and approximately one quarter of the length L (FIG. 5) of the bladder 34. Additionally, such heights H may be, for example, between approximately one eighth and approximately one tenth of a circumference of the sleeve 32. As discussed above, the circumference of the sleeve 32 may be between approximately 9 inches and approximately 15 inches.

As shown in FIG. 2, the elastic panel 36 may be connected at a distance X from the second end 40 of the bladder 34. In exemplary embodiments, the distance X may be between approximately one inch and approximately four inches. Although FIG. 2 illustrates a first end 42 of the elastic panel 36 connected to the first end 38 of the bladder 34 and a second end 44 of the elastic panel 36 connected intermediate the first and second ends 38, 40 of the bladder 34, in additional exemplary embodiments, the second end 44 of the elastic panel 36 may be connected to the second end 40 of the bladder 34. In further embodiments, the distance X may be greater than four inches.

In the exemplary embodiment of FIG. 2, as the distance X increases, the diameter D of the sleeve 32 may correspondingly decrease, and vice versa. The elastic panel 36 may be connected along at least a portion of a width W of the bladder 34, and, as shown in FIG. 2, the elastic panel 36 may be connected to at least one of the ends 38, 40 along the entire width W. In still further exemplary embodiments, a corresponding width of the elastic panel 36 may be greater than or less than the width W.

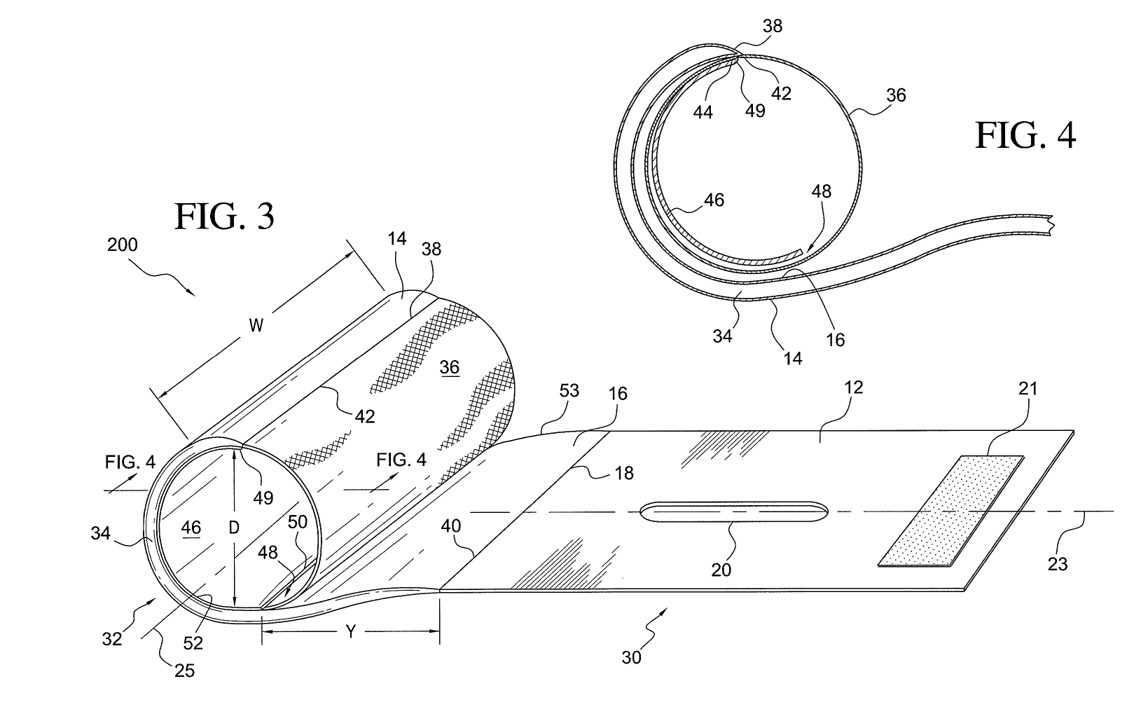

As shown with reference to the blood pressure cuff 200 illustrated in FIGS. 3 and 4, in further exemplary embodiments, a substantially cylindrical sleeve 32 may include an inflatable bladder 34 as described above, a sheet 46 of flexible material connected to at least one of the first and second edges 52, 53 of the bladder 34, and an elastic panel 36 connected to the first end 38 of the bladder 34. In such embodiments, at least the first end 42 of the elastic panel 36 may be connected to the first end 38 of the bladder 34 while the second end 44 of the elastic panel 36 may be connected along the length L (FIG. 5) of the bladder 34 at any distance X (FIG. 2) from the second end 40 of the bladder 34. Alternatively, as shown in FIG. 4, the second end 44 of the elastic panel 36 may be connected proximate the first end 38 of the bladder 34. In still further exemplary embodiments, both ends 42, 44 of the elastic panel 36 may be connected to the first end 38 of the bladder 34.

The elastic panel 36 may be at least partially disposed between the sheet 46 and the bladder 34. For example, the sheet 46 and the inner surface 16 of the bladder 34 may together form a pocket 48, and at least a portion of the elastic panel 36 may be moveably disposed within the pocket 48 between the sheet 46 and the inner surface 16. The sheet 46 may be formed from any of the materials described above with regard to the cover 33. As shown in FIG. 3, a first end 49 of the sheet 46 may be connected to the first end 38 of the bladder 34, and a second end 50 of the sheet 46 may be unconnected to the bladder 34 so as to permit movement of the elastic panel 36 into and out of the pocket 48. In exemplary embodiments, the second end 50 of the sheet 46 may be disposed a distance Y between approximately four inches and approximately eight inches from the second end 40 of the bladder 34. For example, as the elastic panel 36 expands, a portion of the panel 36 may exit the pocket 48, and as the elastic panel 36 contracts, a portion of the panel 36 may enter the pocket 48. In exemplary embodiments, at least a portion of the first end 49 of the sheet 46 may be connected along the width W of the bladder 34 at the first end 38, and in additional exemplary embodiments, the entire first end 49 of the sheet 46 may be connected to the first end 38 along the width W. The sheet 46 may be elastic or inelastic, and the sheet 46 may interface with an arm of the patient as the sleeve 32 is disposed about the arm. In such exemplary embodiments, the interface between the sheet 46 and the arm may facilitate expansion and/or retraction of the elastic panel 36. In such exemplary embodiments, the sheet 46 may comprise between approximately one quarter and approximately three quarters of a circumference of the sleeve 32. In addition, as shown in FIG. 3, the sheet 46 may comprise a portion of an inner wall of the sleeve 32 while the elastic panel 36 comprises a remainder of the inner wall of the sleeve 32.

As shown with reference to the blood pressure cuff 300 illustrated in FIG. 5, in further exemplary embodiments, a substantially cylindrical sleeve 32 may include an elastic panel 36, connected to an inelastic panel 54. Such an exemplary blood pressure cuff 300 may also include an inflatable bladder 34 connected to the sleeve 32 and configured to at least partially encircle at least a portion of the elastic panel 36 and/or the inelastic panel 54. As shown in FIG. 5, in such an exemplary embodiment, the longitudinal axis 23 of the tail 12 may be collinear with a longitudinal axis of the bladder 34, and the tail 12 may be connected to and/or otherwise extend from the second end 40 of the bladder 34. The longitudinal axis of the bladder 34 may extend transverse to the central axis 25 of the sleeve 32.

The first end 38 of the bladder 34 may be connected to the first end 42 of the elastic panel 36 as well as a first end 56 of the inelastic panel 54. A second end 58 of the inelastic panel 54 may be connected to the second end 44 of the elastic panel 36. Although FIG. 5 illustrates the panels 36, 54 as having the same width (i.e., the width W of the sleeve 32), in additional exemplary embodiments, one of the panels 36, 54 may have a different width than the other of the panels 36, 54. Additionally, although FIG. 5 illustrates the panels 36, 54 as comprising substantially equal respective percentages of the circumference of the sleeve 32, in additional exemplary embodiments, one of the panels 36, 54 may comprise a different percentage of the circumference than the other of the panels 36, 54. For example, the elastic panel 36 may comprise a greater percentage of the circumference of the sleeve 32 to assist in increasing the overall flexibility of the sleeve 32, thereby facilitating disposal of the sleeve 32 on and/or removal of the sleeve 32 from an arm of a patient. Such a greater percentage may be, for example, approximately 60 percent or greater.

The inelastic panel 54 may be made from any of the materials described above with regard to the bladder 34. While such materials may be highly flexible, durable, and suitable for contact with, for example, the skin of a patient, the materials used to form the inelastic panel 54 may also be resistant to lateral and/or longitudinal expansion. Such resistance may further facilitate disposal of the sleeve 32 on and/or removal of the sleeve 32 from the arm of the patient. In further exemplary embodiments, the sheet 46 and the inelastic panel 54 shown in FIG. 5 may be a single piece of material that is sealed to the elastic panel 36 along the second end 44 thereof. In still further exemplary embodiments, the sheet 46 may be omitted.

The exemplary embodiments of the present disclosure provide a variety of advantages over known blood pressure cuffs for applications in which patients wish to measure and/or monitor their own blood pressure without receiving assistance from another person. For example, known blood pressure cuffs are only operable to occlude the brachial artery of a patient by wrapping an inflatable bladder of the cuff around the circumference of the patient's upper arm. Once the bladder has been properly positioned, a tail attached to the bladder must then be wrapped around the bladder while holding the bladder in place. The tail may then be releasably attached onto either itself or an outer surface of the bladder, prior to inflation of the bladder. While the standard bladder and tail configuration of known blood pressure cuffs may make it cumbersome for a patient to properly position, wrap, and secure the cuff around his or her own arm without assistance, such difficulties are overcome by the aspects of the disclosed blood pressure cuffs.

For example, each of the blood pressure cuffs disclosed herein include a substantially cylindrical sleeve configured to accept an arm of the patient, and the patient may position the sleeve on his or her upper arm with a single hand. As a result, the sleeves of the present disclosure eliminate the need for holding a first portion (i.e., the bladder) of the blood pressure cuff in place on the upper aim while a second portion (i.e., the tail) is wrapped around the first portion. Additionally, at least a portion of the disclosed cylindrical sleeves, such as the elastic panel 36, may be made from a flexible elastic material. The elasticity provided by such portions is absent from standard blood pressure cuffs, and such elastic portions assist the sleeve in advantageously clinging to the arm of the patient while the disclosed cuffs are initially positioned on the upper arm. Such elastic portions also assist in sliding the sleeve over, for example, the hand and/or elbow by providing local and temporary variability in the diameter and/or circumference of the sleeve.

Although the exemplary blood pressure cuffs of FIGS. 1-5 may be used in accordance with each of the exemplary methods described herein, for ease of discussion, the blood pressure cuff 100 of FIG. 2 will be described for the duration of this disclosure. In addition, for ease of discussion, manipulation of the blood pressure cuff 100 using only a patient's right hand, without assistance, will be discussed below. Similar steps could be performed, however, to manipulate the blood pressure cuff 100 using only the patient's left hand, without assistance.

To position the blood pressure cuff 100 on his or her left arm without assistance, the patient may insert his or her left hand into the sleeve 32 and pull the sleeve 32 up his or her left arm with his or her right hand. Once properly positioned on the patient's upper arm, such as between the patient's armpit and elbow, the elastic panel 36 may assist the sleeve 32 in remaining substantially stationary along the upper arm without further interaction from the patient. The patient may then wrap the tail 12 around the outer surface 14 of the bladder 34 and attach the connector 21 to either the outer surface 14 or an outer surface of the tail. In doing so, the patient may align the slot 20 with the port 22 such that the port 22 protrudes through the slot 20. The patient may then connect, for example, a hand-held inflation bulb (not shown) to the port 22 to assist with inflating the bladder 34. Using the bulb, the patient may inflate the bladder 34 of the blood pressure cuff 100 to at least 80 mm Hg, above which the brachial artery within the patient's arm may become occluded. Occluding the brachial artery may block a flow of blood from the heart through the artery.

While the bladder 34 is in such an inflated state, the blood pressure of the patient may be measured utilizing, for example, a sphygmomanometer and a stethoscope, and/or other like devices. For example, the blood pressure of the patient may be measured in conjunction with slowly releasing fluid from the blood pressure cuff. The pressure at which the sound of blood flow within the brachial artery can first be heard may be measured (systolic blood pressure), and as the fluid continues to exit the cuff, the pressure may again be measured when no sound within the artery can be heard (diastolic blood pressure). Once all measurements have been taken, and the bladder 34 has been completely deflated, the patient may remove the cuff 100 from his or her arm, without assistance, by disengaging the connector 21 from the outer surface 14 of the bladder 34 and/or from the outer surface of the tail 12. Once the connector 21 is disengaged, the patient may slide the sleeve 32 from the upper arm down past the patient's hand.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A blood pressure cuff, comprising:
a substantially cylindrical sleeve configured to accept an arm of a patient, the sleeve including
an inflatable bladder having a first end, and a second end disposed opposite the first end, and
an elastic panel connected intermediate the first and second ends of the bladder; and
a tail connected to the second end of the bladder, the tail including a longitudinal axis disposed transverse to a central axis of the sleeve.

2. The blood pressure cuff of claim 1, wherein the elastic panel has a height between approximately one eighth and approximately one quarter of a length of the bladder.

3. The blood pressure cuff of claim 1, wherein the elastic panel is connected between approximately one inch and approximately four inches from the second end of the bladder.

4. The blood pressure cuff of claim 1, wherein an inner surface of an exterior of the bladder comprises a portion of an inner wall of the sleeve and the elastic panel comprises a remainder of the inner wall.

5. The blood pressure cuff of claim 1, wherein a first end of the elastic panel is connected to the first end of the bladder, and a second end of the elastic panel is connected intermediate the first and second ends of the bladder.

6. A blood pressure cuff, comprising:
a substantially cylindrical sleeve configured to accept an arm of a patient, the sleeve including
an inflatable bladder having a first edge, a second edge disposed opposite the first edge, and a first end disposed transverse to the first and second edges,
a flexible sheet connected along a portion of the first and second edges of the bladder, and
an elastic panel connected to the first end of the bladder, at least a portion of the elastic panel being disposed between the sheet and the bladder.

7. The blood pressure cuff of claim 6, wherein a first end of the sheet is connected to the first end of the bladder.

8. The blood pressure cuff of claim 7, wherein a second end of the sheet is disposed between approximately four inches and approximately eight inches from a second end of the bladder.

9. The blood pressure cuff of claim 6, wherein the sheet comprises between approximately one quarter and approximately three quarters of a circumference of the sleeve.

10. The blood pressure cuff of claim 6, wherein the sheet comprises a portion of an inner wall of the sleeve and the elastic panel comprises a remainder of the inner wall of the sleeve.

11. The blood pressure cuff of claim 6, wherein a second end of the elastic panel is connected proximate the first end of the bladder.

12. A blood pressure cuff, comprising:
a substantially cylindrical sleeve configured to accept an arm of a patient, the sleeve including an elastic panel, and an inelastic panel connected to the elastic panel along a width of the sleeve;
a flexible sheet connected to the inelastic panel;
an inflatable bladder connected to the sleeve, the bladder having a longitudinal axis disposed transverse to a central axis of the sleeve; and
a tail connected to the bladder, the tail including a longitudinal axis disposed collinear with a longitudinal axis of the bladder and transverse to a central axis of the sleeve.

13. The blood pressure cuff of claim 12, wherein the bladder is connected to a first end of the inelastic panel, and the elastic panel is connected to a second end of the inelastic panel opposite the first end.

14. The blood pressure cuff of claim 12, wherein the inelastic panel has a first width and the elastic panel has a second width different than the first width.

15. The blood pressure cuff of claim 12, wherein the elastic and inelastic panels comprise substantially equal percentages of a circumference of the sleeve.

16. A blood pressure cuff, comprising:
a substantially cylindrical sleeve including a cover and an inflatable bladder disposed within the cover,
an exterior of the cover having an outer wall and in inner wall, wherein the outer wall includes a first connector and the inner wall is configured to accept an arm of a patient; and
a tail connected to the sleeve, the tail including a second connector releasably attachable to the first connector.

17. The blood pressure cuff of claim 16, further including a port fluidly connected to the bladder.

18. The blood pressure cuff of claim 17, wherein the port is connected to a tube passing through the cover, the tube fluidly connecting the port to the bladder.

19. The blood pressure cuff of claim 16, wherein the cover comprises an elastic material and the first connector comprises an inelastic material.

20. The blood pressure cuff of claim 16, wherein the cover comprises an expandable inner cavity and the bladder is disposed within the inner cavity, the cavity maintaining the bladder in a substantially cylindrical shape.
